(19) United States
(12) Patent Application Publication
(10) Pub. No.: US 2024/0068988 A1
(43) Pub. Date: Feb. 29, 2024

(54) SYSTEMS AND METHODS FOR RAPID PATHOGEN DETECTION

(71) Applicant: Darrell D. Marshall, Detroit, MI (US)

(72) Inventor: Darrell D. Marshall, Detroit, MI (US)

(21) Appl. No.: 18/240,800

(22) Filed: Aug. 31, 2023

Related U.S. Application Data
(60) Provisional application No. 63/374,172, filed on Aug. 31, 2022.

(51) Int. Cl. G01N 27/622; G16B 40/00

(52) U.S. Cl. CPC G01N 27/622; G16B 40/00

(57) ABSTRACT

A device for detecting pathogens comprises a chamber configured to receive a sample including an analyte and a matrix, a gas inlet extending into the chamber and configured to adjust the pressure within the chamber to ionize the volatile or non-volatile molecules of the sample, an ion mobility spectrometer configured to obtain the ionized molecules of the sample to obtain pathogen data, and a computing device configured to analyze the pathogen data to determine one or more pathogens of the analyte.

### CROSS REFERENCE TO RELATED APPLICATIONS

This U.S. patent application claims priority under 35 U.S.C. § 119(e) to U.S. Provisional Application 63/374,172, filed on Aug. 31, 2022. The disclosure of this prior application is considered part of the disclosure of this application and is hereby incorporated by reference in its entirety.

### TECHNICAL FIELD

This disclosure relates to systems and methods for rapid microorganism, environmental toxin, or pathogen detection.

### BACKGROUND

Today, the global population's accessibility to transportation connectivity has increased the mobility of people and, as a result, the mobility of pathogen-related infections. The COVID-19 pandemic highlighted many issues with the global biodefense infrastructure, and the lack of rapid pathogen identification played a significant role in the spread of COVID-19. Some of these issues include struggling or failing to provide rapid onsite pathogen identifications, and, instead, many agencies and entities rely on slow and costly offsite testing, business shutdowns, and mass-scale quarantines, which could indirectly be a factor in infection proliferation. Thus, a reliable, rapid onsite pathogen screening is needed to respond quickly to pathogen outbreaks or biological warfare agent incidents efficiently.

### SUMMARY

One aspect of the disclosure provides a device for detecting pathogens comprising a chamber configured to receive a sample including an analyte and a matrix, a gas inlet extending into the chamber and configured to adjust the pressure within the chamber to ionize the molecules of the sample, an ion mobility spectrometer configured to obtain the ionized molecules of the sample to obtain pathogen data, and a computing device configured to analyze the pathogen data to determine one or more pathogens of the analyte.

Implementations of the disclosure may include one or more of the following optional features. In some implementations, the device further comprises a housing configured to house the chamber, the ion mobility spectrometer, and the computing device. The housing may be configured to be handheld.

The device may further comprise a heat source within the chamber configured to adjust the temperature within the chamber.

The computing device may analyze the pathogen data using artificial intelligence, which may include deep learning algorithms to improve the robustness of the analysis of the pathogen data.

The device may further comprise an airlock door configured to selectively permit access to the chamber. The device may further comprise a sampling rod configured to selectively transmit the sample into the chamber.

Another aspect of the disclosure provides a system for detecting pathogens comprising a pathogen detection device comprising a chamber configured to receive a sample including an analyte and a matrix, a gas inlet extending into the chamber and configured to adjust the pressure within the chamber to ionize the molecules of the sample, an ion mobility spectrometer configured to obtain the ionized molecules of the sample to obtain pathogen data, and an external computing device in communication with the pathogen detection, the external computing device configured to analyze the pathogen data to determine one or more pathogens of the analyte.

This aspect may include one or more of the following optional features. In some implementations, the external computing device is one of a smartphone, a laptop computer, a desktop computer, or a tablet computer.

The pathogen detection device may be in communication with the external computing device via a wireless telecommunication network.

The external computing device may include data processing hardware and memory hardware in communication with the data processing hardware, the memory hardware storing instructions that when executed on the data processing hardware cause the data processing hardware to perform operations to analyze the pathogen data to determine one or more pathogens of the analyte. The operations may include analyzing the pathogen data using artificial intelligence.

The external computing device may be in communication with cloud computing resources storing a pathogen database including a plurality of deep learning algorithms and a plurality of pathogen fingerprint profiles.

Another aspect of the disclosure provides a method for detecting pathogens comprising introducing a sample into a pathogen detection device, adjusting the pressure and temperature within the pathogen detection device to ionize the molecules of the sample, introducing the ionized molecules of the sample into an ion mobility spectrometer, operating the ionized mobility spectrometer, obtaining pathogen data from the ionized mobility spectrometer related to the ionized molecules of the sample, analyzing the pathogen data, and determining at least one pathogen of the sample based on the analysis of the pathogen data.

The pathogen detection device and the ion mobility spectrometer may be contained within the same housing.

The pressure within the pathogen detection device may be adjusted via a gas inlet into the pathogen detection device.

The method may further comprise adjusting the temperature within the pathogen detection device via one of an ultraviolet lamp or an infrared lamp.

The pathogen data may be analyzed using artificial intelligence. The pathogen data may be analyzed using one or more deep learning algorithms to improve the robustness of the analysis of the pathogen data.

The details of one or more implementations of the disclosure are set forth in the accompanying drawings and the description below. Other aspects, features, and advantages will be apparent from the description and drawings, and from the claims.

### DESCRIPTION OF DRAWINGS

Reference will now be made to the accompanying Figures, which are not necessarily drawn to scale, and wherein:

FIG. 1A is a perspective view of an exemplary matrix-assisted ionization (MAI) device in accordance with principles of the present disclosure;

FIG. 1B is a cross-sectional perspective view of the MAI device of FIG. 1A, taken along lines 1B-1B;

FIG. 2A is a schematic view of the MAI device of FIG. 1A and an exemplary ion mobility spectrometry (IMS) device in accordance with principles of the present disclosure;

FIG. 2B is a schematic view of an exemplary MAI-IMS device which incorporates the MAI device of FIGS. 1A-1B and the IMS device of FIG. 2A;

FIG. 3 is a method for use of the MAI-IMS device of FIG. 2B;

FIG. 4 is a schematic view of a system including the MAI-IMS device of FIG. 2B.

Like reference symbols in the various drawings indicate like elements.

### DETAILED DESCRIPTION

Some implementations of the disclosed technology will be described more fully with reference to the accompanying drawings. This disclosed technology may, however, be embodied in many different forms and should not be construed as limited to the implementations set forth herein.

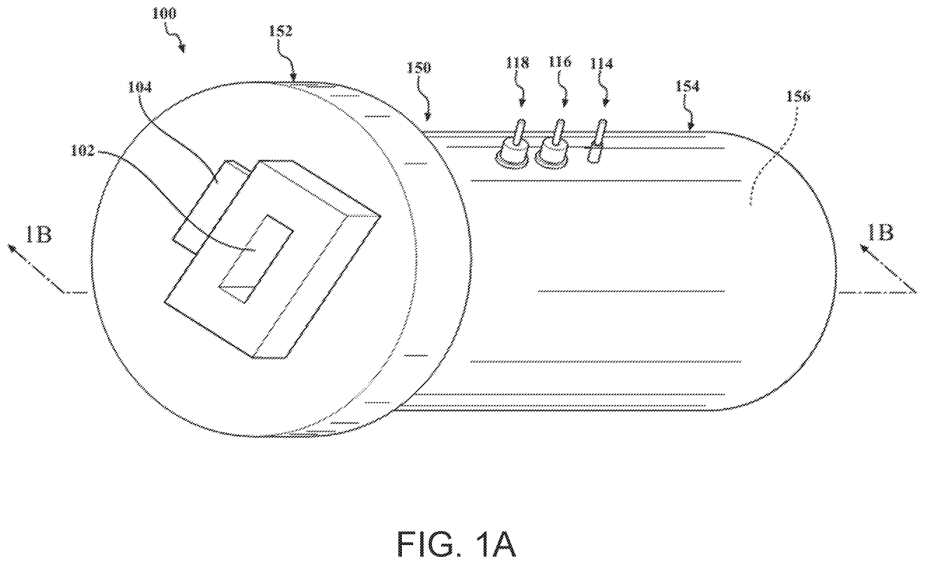

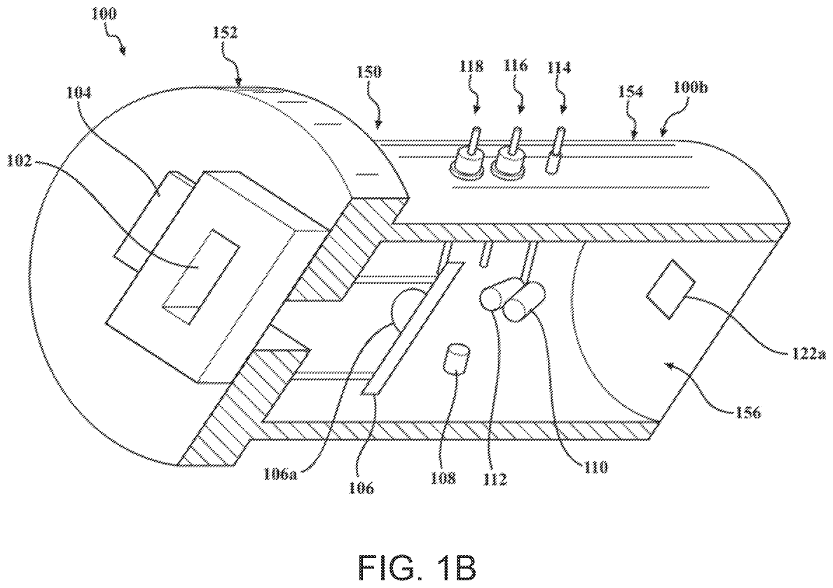

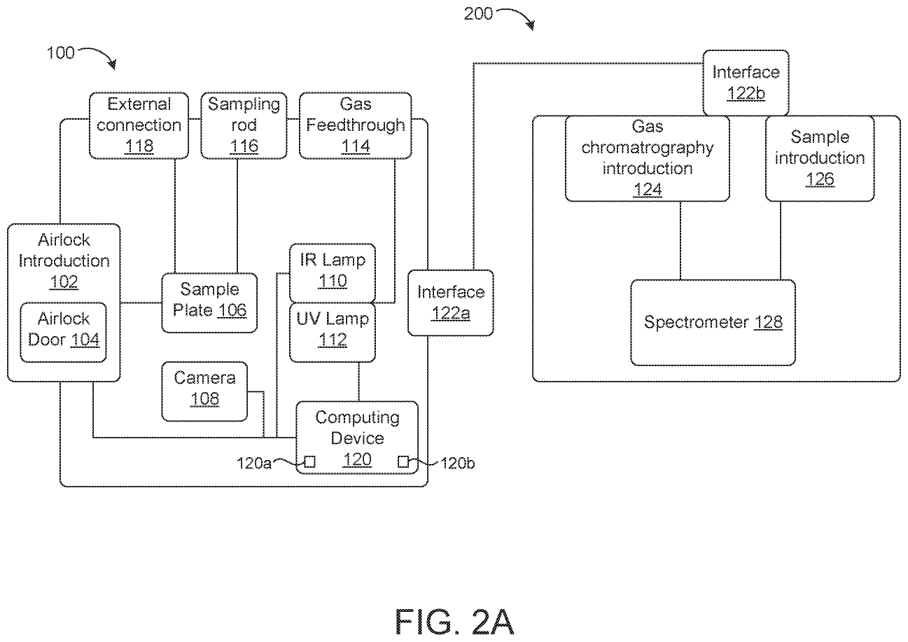

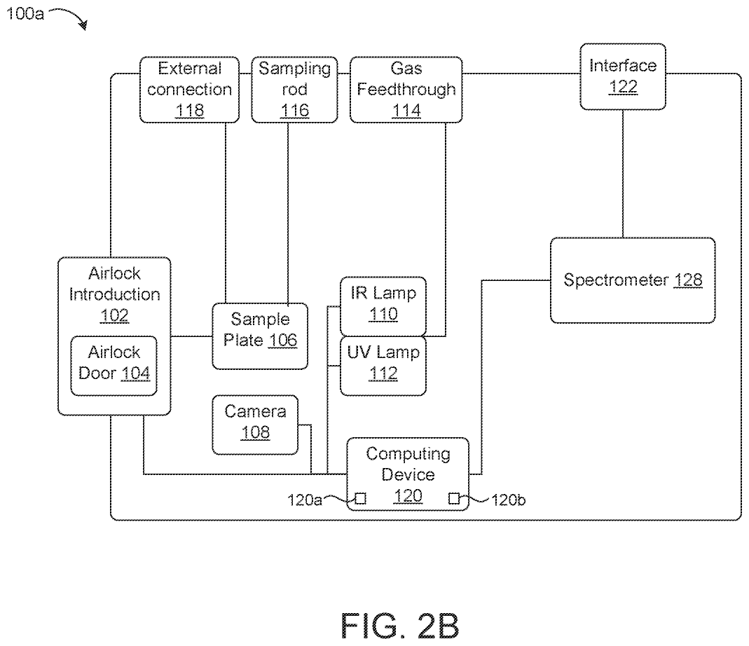

Referring to FIGS. 1A-1B, a matrix-assisted ionization (MAI) device 100 is generally shown. In some implementations, the MAI device 100 may interface with (or retrofit) an ion mobility spectrometry (IMS) device 200, as shown in FIG. 2A. In other implementations, as shown in FIG. 2B, an MAI-IMS device 100a may incorporate the MAI device 100 of FIGS. 1A-1B and the IMS device 200 of FIG. 2A.

The MAI device 100 may use a mixture of an analyte and a matrix in solution and may not require a laser. To ionize the analyte, the MAI device 100 may apply a pressure differential and specific matrix to the solution, spontaneously converting all classes of volatile and non-volatile biomolecules to gas-phase ions. In some implementations, the matrix may include any suitable solvent, such as 2-aminobenzyl alcohol, anthranilic acid, 2-hydroxyacetophenone, 1,1-Dimethylpyrrolidine-2,5-dione (DMPD), 1,2-Dicyanobenzene (1,2-DCB), 1,5-Diaminonaphthalene (1,5-DAN), 1,5-Naphthalenediamine (1,5-ND), 2-Amino-5-nitropyridine (2A5NP), 2,4-Dihydroxybenzylamine (DHBAm), 2,4,6-Trihydroxyacetophenone (THAP), 2,5-Dihydroxybenzoic acid (DHB), 2,5-Dihydroxybenzylamine (DHBA), 2,6-Dihydroxyacetophenone (DHAP), 3-Hydroxypicolinic acid (3-HPA), 3-Nitrobenzonitrile (3-NBN), 3,4-Dihydroxybenzaldehyde (3,4-DHBA), 4-Nitroacetanilide (4-NAA), 6-Aminoquinoline (6-AQ), 6-Aza-2-thiothymine (ATT), 7-Azaindole (7AI), 7,7,8,8-Tetracyanoquinodimethane (TCNQ), 9-Aminoacridine (9-AA), 9,10-Dihydroxyanthracene (DHA), Benzoyl chloride (BzC1), Bis(dimethylamino)naphthalene (DMAN), Cinnamic Acid Derivatives, Dihydroxybenzoic acid (DHB), Ferulic acid, Ferulic Acid (FA), Nitroaromatic Compounds, p-Nitroaniline (pNA), Sinapinic acid (SA), or α-Cyano-4-hydroxycinnamic acid (CHCA). Accordingly, the MAI device 100 may require less power input demand than other systems as it does not require high voltages or lasers for ionization.

The MAI device 100 may allow for the ionization of small and large biologically relevant molecules, such as lipids, peptides, proteins, deoxyribonucleic acids, ribonucleic acids, carbohydrates, metabolites, or their derivatives, etc., to produce a molecular fingerprint of a microorganism or environmental toxin. The ionization by the MAI device 100 may include reduced insource fragmentation products, which may be referred to as a soft ionization technique, and which may allow the MAI device 100 to analyze delicate molecules.

The IMS device 200 may be relatively compact, portable, and widely available. The gas-phase ionization of the IMS device 200 may occur by bombarding volatile molecules with high-energy electrons from Ni beta-rays. The IMS device 200 may create an electric field that transfers the ions into a drift tube filled with an inert gas. The IMS device 200 may separate and identify gas-phase ions based on their gas collisions rates calculated from mobility, size, and mass-to-charge ratio.

In some implementations, the IMS device 200 may include, or be coupled with, multi-capillary columns (MCC-IMS) to increase the resolution and provide a 2D mobility plot. In such implementations, the IMS device 200 may provide an orthogonal dimension of separation to increase the resolution. In other implementations, the IMS device 200 may use Helium in the drift gas mixture to increase the resolution of complex mixtures for biomolecular analyses. In yet other implementations, the IMS device 200 may be a trapped ion mobility spectrometry (TIMS) to increase resolution.

The MAI-IMS device 100a combines MAI and IMS to allow for spontaneously converting all classes of volatile and nonvolatile biomolecules to gas-phase ions. The MAI-IMS device 100a may receive a sample of interest 106a mixed with an MAI matrix and create a differential pressure region (e.g., atmospheric pressure (AP) to vacuum pressure), promoting sublimation of the sample 106a, i.e., matrix mix producing gas-phase ions. The pressure differential in the MAI-IMS device 100a may cause disruptions in the sample 106a, i.e., matrix crystals resulting in matrix sublimation causing micro “explosions” due to the reduced surface area from the matrix converting to the gas-phase ions. This may result in a charge separation process, e.g., fracto/triboluminescence, which is the release of energy from the breaking of crystals, so that particles are displaced into the gas phase having an excess positive or negative charge. Therefore, an external energy source (via laser, voltage, electronics, or other bulky, expensive equipment) may not be needed for ionization to occur.

Referring to FIGS. 1A and 1B, the MAI device 100 may include a housing 150 having an introduction portion 152 and a body portion 154, the body portion 154 defining an interior chamber 156. The housing 150 may be formed from any suitable metal, such as steel, iron, stainless steel, etc., or plastic, such as polyethylene terephthalate, low or high density polyethylene, polyvinyl chloride, polypropylene, bisphenol A, etc. The housing 150 shown in FIGS. 1A and 1B is exemplary only, and it should be understood that the housing 150 may include any suitable shape and configuration. In some implementations, the housing 105 may be designed to be handheld, e.g., it may be less than the size of a traditional textbook.

The MAI device 100 includes an airlock introduction port 102 including an airlock door 104. The airlock door 104 may be automatically operated or manually operated. The airlock introduction 102 facilitates access to a sample plate 106 in the chamber 156 that is configured to receive the sample 106a, which includes an analyte of interest and any suitable matrix, as set forth above. In some implementations, the sample 106a may be introduced into the MAI device 100 via a matrix mix inserted into the MAI device 100, or via a sampling rod 116. In implementations where the sample rod 116 is used to introduce the sample 106a, a swab, a test strip, or other suitable mechanism may be used to facilitate introduction of the sample 106a into the device 100. In some implementations, the swab or strip may be reusable.

The MAI device 100 may include a camera 108, an infrared (IR) lamp 110, and an ultraviolet (UV) lamp 112 in the chamber 156. The camera 108 may be a high-speed camera or any other suitable camera. The camera 108 may provide a live video feed to a user (e.g., an electronic device of the user as set forth below) of the sample 106a to ensure that the sample 106a is being ionized and that the sample 106a is at the correct location.

The IR lamp 110 and the UV lamp 112 are configured to adjust the heat in the MAI 100 to desired levels. In some implementations, the IR lamp 110 and the UV lamp 112 may adjust the temperature of the chamber 156 to between approximately 40° C. and 150° C. The MAI device 100 includes a gas feedthrough 114 configured to adjust the inert or other suitable gas pressure in the MAI device 100 to desired levels. In some implementations, the gas feedthrough 114 may adjust the pressure in the chamber 156 to between approximately 1.0×10⁻³ torr and 1.0×10³ torr. The gas feedthrough 114 may receive external gas connections to modify the pressure within the MAI device 100. Additionally, the MAI device 100 may include an external connection 118 to facilitate any additional connections as desired. For example, the external connection 118 may be configured to receive electronic devices, voltage applications, or any other suitable external connection.

The MAI device 100 may separate and concentrate pathogens of the sample 106a by electrokinetic concentration, which, in some implementations, may perform isolation and concentration of bacteria in approximately three minutes, and the density factor may be increased nearly a thousand-fold in a local area of approximately 5000 μm² from a low bacteria concentration of 5×10³ CFU/ml. In some implementations, the MAI device 100 may be connected with capillary electrophoresis (CE) systems, microfluidic devices, lab-on-a-chip systems, ion-selective membrane devices, electrochemical sensors, field-flow fractionation, electrophoretic devices, etc.

The MAI device 100 may include a computing device 120 including data processing hardware 120a (e.g., a computing device that executes instructions) and memory hardware 120b. The memory hardware 120b is in communication with the data processing hardware 120a and the memory hardware 120b stores instructions that are executed by the data processing hardware 120a. For example, the data processing hardware 120a may execute instructions that control one or more of the electromechanical devices of the MAI device 100, including, but not limited to the airlock introduction 102, the airlock door 104, the sample plate 106, the camera 108, the IR lamp 110, the UV lamp 112, the gas feedthrough 114, the sampling rod 116, the external connection 118, and a spectrometer 128 (for the MAI-IMS device 100a).

Referring to FIGS. 1A-1B and 2A, the MAI device 100 may be in communication with the IMS device 200 via a first interface 122a in the MAI device 100 and a second interface 122b in the IMS device 200. For example, the IMS device 200 may include the spectrometer 128, a sample introduction 126, and a gas or liquid chromatography introduction 124. In some implementations, the sample 106a may pass through a liquid chromatography system before the sample 106a is introduced into the MAI device 100. In some implementations, the sample 106a may pass through a gas chromatography system before the sample 106a is introduced into the IMS device 200. The second interface 122b may receive the sample 106a from the first interface 122a. Such transmission of the sample 106a may be performed automatically via any suitable means, i.e., mechanical, electro-mechanical, etc., or the transmission may be performed manually, e.g., by a user. The first interface 122a may be an ion gate that opens and closes to allow gas ions to flow into the spectrometer 128 via the combination of carrier gas and electric field from a low voltage extraction of the ion optic focusing.

Referring to FIG. 2B, the MAI-IMS device 100a may incorporate the spectrometer 128 within the confines of device 100a itself. The foregoing description of the MAI device 100 and the IMS device 200 as two separate components, may equally apply to the MAI-IMS device 100a as one single component. In some implementations, the MAI-IMS device 100a may receive the sample 106a through the airlock door 104 or the sampling rod 116, and then, after ionization, the gas ions of the sample 106a may be introduced to the spectrometer 128 via the interface 122. In other implementations, the sample 106a may be introduced to the MAI-IMS device 100a via the airlock door 104 or the sampling rod 116, and then, after ionization is complete, the ionized gas molecules of the sample 106a may be automatically transferred to the spectrometer 128.

The spectrometer 128 may use any suitable analysis, such as drift tube IMS, low pressure drift tube IMS, travelling wave IMS, trapped IMS, high-field asymmetric waveform IMS, differential mobility analyzer, etc., to determine MAI-IMS data related to the ionized gas molecules of the sample 106a. The computing device 120 may obtain the MAI-IMS data from the spectrometer 128 to obtain a molecular fingerprint of the microorganism(s) of the sample 106a and determine the pathogen(s) and other microorganisms of the sample 106a.

In some implementations, the MAI-IMS device 100a may be in communication with an external computing device 420, such as a standard server 320a, as a laptop computer 320b, as part of a rack server system 320c, as a mobile device 320d (such as a smartphone), or as a tablet computer 320e, etc., such that the computing device 120 resides in one or more of the external computing devices 420. For example, the MAI-IMS device 100a may be incorporated into a smartphone case and is in communication with the smartphone contained in the case (e.g., wired or wireless connection), wherein the smartphone includes the computing device 120 that performs one or more of the functions described above.

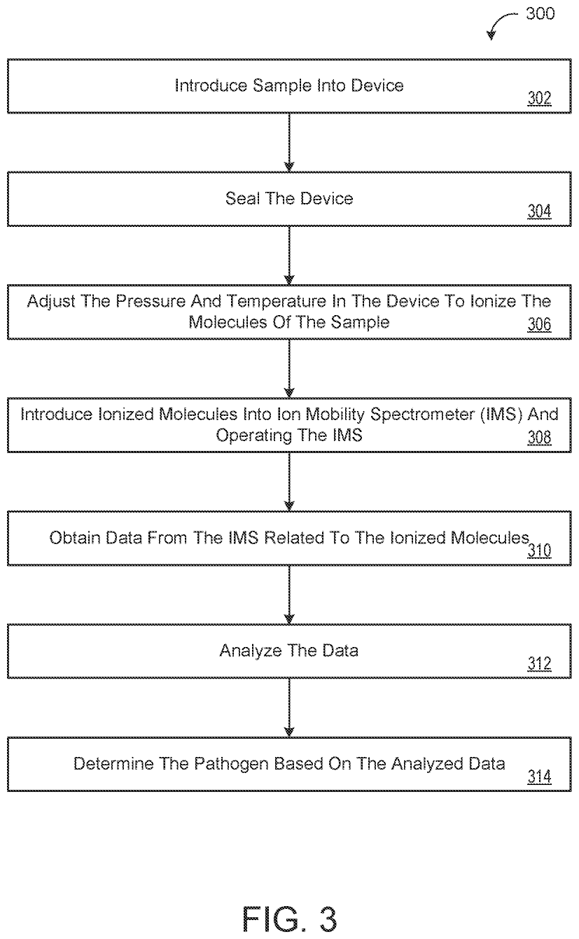

Referring to FIG. 3, a method 300 for operating the MAI-IMS device 100a is generally shown. At step 302, the sample 106a is introduced into the chamber 156 of the MAI-IMS device 100a, e.g., via the airlock door 104 and/or the sampling rod 116. At step 304, the MAI-IMS device 100a is sealed, e.g., by closing and sealing the airlock door 104. At step 306, the IR lamp 110 and the UV lamp 112 adjust the heat within the chamber 156 to the desired temperature and the gas feedthrough 114 and/or the external connection 118 adjust the pressure within the chamber 156 to the desired temperature to ionize the molecules of the sample 106a. In some implementations, no heat is required.

At step 308, the ionized molecules are transmitted to the ion mobility spectrometer 128 and the ion mobility spectrometer 128 is operated by the ionized molecules being accelerated through a drift field to a detector. At step 310, the computing device 120 obtains the MAI-IMS data from operation of the ion mobility spectrometer 128. At step 312, the computing device 120 analyzes the MAI-IMS data. Such analysis may include comparing the MAI-IMS data to pathogen fingerprint profiles 454, 454a-n, implementing artificial intelligence such as conducting one of the deep learning algorithms 452, 452a-n, or any other suitable analysis. At step 314, the computing device 120 determines the pathogen based on the analyzed MAI-IMS data.

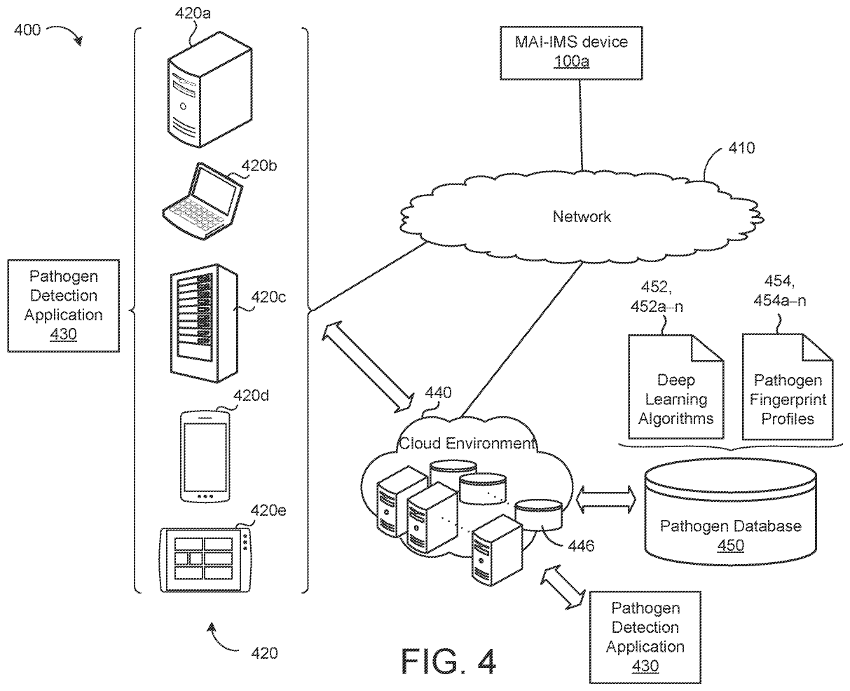

Referring to FIG. 4, a system 400 for implementing the MAI-IMS device 100a is generally shown. The system 400 may include a network 410 which provides access to cloud computing resources 440 (e.g., distributed system) for providing access to a pathogen database 450. The system 400 may include a pathogen detection application 430 that may be executed by a number of different devices 420, including a standard server 420a, as a laptop computer 420b, as part of a rack server system 420c, as a mobile device 420d (such as a smartphone), or as a tablet computer 420e. In some implementations, all or a portion of the pathogen detection application 430 may be executed by the cloud computing resources 440. The pathogen detection application 430 may be configured to obtain and analyze data from the MAI-IMS device 100a in order to determine whether the sample 106a includes any pathogens, which pathogens the sample 106a may include, origin of the pathogen, variant and strain analysis, etc.

The network 410 may include any type of network that allows sending and receiving communication signals, such as a wireless telecommunication network, the Internet, a cellular telephone network, a time division multiple access (TDMA) network, a code division multiple access (CDMA) network, Global system for mobile communications (GSM), a third generation (3G) network, fourth generation (4G) network, fifth generation (5G) network, a satellite communications network, and other communication networks. The network 410 may include one or more of a Wide Area Network (WAN), a Local Area Network (LAN), and a Personal Area Network (PAN). In some examples, the network 410 includes a combination of data networks, telecommunication networks, and a combination of data and telecommunication networks. The MAI-IMS device 100a, the device 420 (including the pathogen detection application 430), and the pathogen database 450 communicate with each other by sending and receiving signals (wired or wireless) via the network 410, which, in some examples, may utilize Bluetooth, Wi-Fi, etc. In some examples, the network 410 provides access to cloud computing resources, which may be elastic/on-demand computing and/or storage resources 446 available over the network 410. The term “cloud” services generally refers to a service delivered from one or more remote devices accessible via one or more networks 410, rather than a service performed locally on a user's device.

The pathogen database 450 may store a plurality of deep learning algorithms 452, 452a-n and a plurality of pathogen fingerprint profiles 454, 454a-n. The pathogen database 450 may be customizable with the deep learning algorithms 452, 452a-n to identify, increase, improve robustness of, and expand the pathogen fingerprint profiles 454, 454a-n to decrease operator error, resulting in the MAI-IMS device 100a being an ideal portable device for universal pathogen onsite screening.

The pathogen detection application 430 used to classify microorganisms may be based on multivariate statistics including, but not limited to, e.g., principal component analysis (PCA) to generate PCA data, orthogonal projections to latent structures discriminant analysis (OPLS-DA) to generate OPLS-DA data, random forests (RF) to generate RF data, and/or library matching to generate library matching data. The PCA may be an unsupervised technique for visualizing variance in the observations. For example, classification may not be defined and may depend on spectral variance to the group. The pathogen detection application 430 using PCA may reduce the dimensionality of the complex spectral features allowing them to be visualized in a 2D or 3D plot.

OPLS-DA, as implemented by the pathogen detection application 430, may be a supervised technique in which group classification may be manually set. As just two examples, the pathogen detection application 430, utilizing OPLS-DA, may manually set the group classification for Mycobacterium smegmatis (M. smeg) MC² 155 and Escherichia coli (E. coli) K-12. The pathogen detection application 430 using OPLS-DA may identify which variables (molecular features) from the MAI-IMS fingerprints are causing the discrimination between the two classes.

RF, as implemented by the pathogen detection application 430, may be a supervised technique used as an additional classification method. In some implementations, the pathogen detection application 430 may use loadings from the PCA data and raw MAI-IMS data collected from one of the bacteria as a training set to compare these data sets against the remaining observations to speed up the model.

Library matching, as implemented by the pathogen detection application 430, may be done for each bacterial fingerprint separately. In some implementations, the pathogen detection application 430 may use a penalized non-negative linear regression framework using the limited samples for species-specific prototypes, which may be derived directly from the routine reference database of pure spectra.

In some implementations, the pathogen detection application 430 may train the plurality of deep learning algorithms 452, 452a-n using the following exemplary process based on at least one of the pathogen fingerprint profiles 454, 454a-n, the MAI-IMS data collected from the sample 106a, the PCA data, the OPLS-DA data, the RF data, and the library matching data.

The pathogen detection application 430 may obtain the MAI-IMS data from the sample 106a and pre-process the MAI-IMS data by noise removal, normalization, scaling, and data alignment. The pathogen detection application 430 may then perform PCA on the pre-processed MAI-IMS data to visualize the clustering of microorganisms. The pathogen detection application 430 may then train the OPLS-DA process and the RF process by splitting the pre-processed MAI-IMS data into training and test sets. The pathogen detection application 430 may then fit the models of the OPLS-DA process and the RF process to the training data. During the training, the pathogen detection application 430 may select variables or features that cause classification. The pathogen detection application 430 may use the models to predict the identity of unknown microorganisms. The pathogen detection application 430 may evaluate the model's performance using accuracy, precision, recall, and F1-score. The pathogen detection application 430 may implement OPLS-DA and RF processes to prevent overfitting and assess stability.

The pathogen detection application 430 may implement the deep learning algorithms 452, 452a-n using convolutional neural networks (CNNs) to analyze the MAI-IMS generated datasets of microorganism/pathogen fingerprints. In some implementations, the pathogen detection application 430 may obtain the MAI-IMS data from the sample 106a and pre-process the MAI-IMS data by noise removal, baseline correction, peak alignment, scaling, and normalization to correct for instrument performance and sample preparation variations. The pathogen detection application 430 may use the MAI-IMS data to extract relevant features and use them as inputs for the CNNs. The pathogen detection application 430 may select the relevant features using PCA. The pathogen detection application 430 may use a 1D CNN and train the 1D CNN using the pre-processed MAI-IMS data using stochastic gradient descent to optimize the network parameters. The pathogen detection application 430 may validate the model's performance to assess accuracy, precision, and robustness by examining metrics such as recall and F1-score.

The MAI-IMS device 100a allows samples to be spontaneously ionized with an addition of a particular MAI matrix when exposed to the vacuum of the mass spectrometer 10 via a heated inlet tube of the sampling rod 116. A micro-droplet of a matrix (˜0.2 μL) may be exposed to the vacuum at the aperture of the spectrometer 10 inlet. The differential pressure created when introducing a sample into the inlet (1.0 atm to 1.0×10-3 atm) and heat (40° C. to 150° C.) may promote the matrix-assisted ionization process. However, in some implementations, a heat source may not be necessary.

In some implementations, the MAI-IMS device 100a may be used in extraterrestrial applications. For example, the MAI-IMS device 100a may be used to analyze terrestrial or nonterrestrial samples while in outer space, on satellites, on space stations, etc. In other implementations, the MAI-IMS device 100a may be used in military and defense applications, such as bioterrorism threats or other concerns. In other implementations, the MAI-IMS device 100a may be used in transportation applications, such as airports, train stations, shipping docks, etc. In other implementations, the MAI-IMS device 100a may be used in environmental applications, e.g., in remote areas where accessibility to pathogen detection devices may be limited.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

What is claimed is:

1. A device for detecting pathogens comprising:
a chamber configured to receive a sample including an analyte and a matrix;
a gas inlet extending into the chamber and configured to adjust the pressure within the chamber to ionize the molecules of the sample;
an ion mobility spectrometer configured to obtain the ionized molecules of the sample to obtain pathogen data; and
a computing device configured to analyze the pathogen data to determine one or more pathogens of the analyte.

2. The device of claim 1, further comprising a housing configured to house the chamber, the ion mobility spectrometer, and the computing device.

3. The device of claim 2, wherein the housing is configured to be handheld.

4. The device of claim 1, further comprising a heat source within the chamber configured to adjust the temperature within the chamber.

5. The device of claim 1, wherein the computing device analyzes the pathogen data using artificial intelligence.

6. The device of claim 5, wherein the artificial intelligence includes deep learning algorithms to improve the robustness of the analysis of the pathogen data.

7. The device of claim 1, further comprising an airlock door configured to selectively permit access to the chamber.

8. The device of claim 1, further comprising a sampling rod configured to selectively transmit the sample into the chamber.

9. A system for detecting pathogens comprising:
a pathogen detection device comprising:
a chamber configured to receive a sample including an analyte and a matrix;
a gas inlet extending into the chamber and configured to adjust the pressure within the chamber to ionize the molecules of the sample;
an ion mobility spectrometer configured to obtain the ionized molecules of the sample to obtain pathogen data; and
an external computing device in communication with the pathogen detection, the external computing device configured to analyze the pathogen data to determine one or more pathogens of the analyte.

10. The system of claim 9, wherein the external computing device is one of a smartphone, a laptop computer, a desktop computer, or a tablet computer.

11. The system of claim 9, wherein the pathogen detection device is in communication with the external computing device via a wireless telecommunication network.

12. The system of claim 9, wherein the external computing device includes data processing hardware and memory hardware in communication with the data processing hardware, the memory hardware storing instructions that when executed on the data processing hardware cause the data processing hardware to perform operations to analyze the pathogen data to determine one or more pathogens of the analyte.

13. The system of claim 12, wherein the operations include analyzing the pathogen data using artificial intelligence.

14. The system of claim 9, wherein the external computing device is in communication with cloud computing resources storing a pathogen database including a plurality of deep learning algorithms and a plurality of pathogen fingerprint profiles.

15. A method for detecting pathogens comprising:
introducing a sample into a pathogen detection device;
adjusting the pressure within the pathogen detection device to ionize the molecules of the sample;
introducing the ionized molecules of the sample into an ion mobility spectrometer;
operating the ionized mobility spectrometer;
obtaining pathogen data from the ionized mobility spectrometer related to the ionized molecules of the sample;
analyzing the pathogen data; and
determining at least one pathogen of the sample based on the analysis of the pathogen data.

16. The method of claim 15, wherein the pathogen detection device and the ion mobility spectrometer are contained within the same housing.

17. The method of claim 15, wherein the pressure within the pathogen detection device is adjusted via a gas inlet into the pathogen detection device.

18. The method of claim 15, further comprising adjusting the temperature within the pathogen detection device via one of an ultraviolet lamp or an infrared lamp.

19. The method of claim 15, wherein the pathogen data is analyzed using artificial intelligence.

20. The method of claim 19, wherein the pathogen data is analyzed using one or more deep learning algorithms to improve the robustness of the analysis of the pathogen data.
